(19) United States
(12) Patent Application Publication
(10) Pub. No.: US 2024/0423550 A1
(43) Pub. Date: Dec. 26, 2024

(54) Concussion Detection System and Method of Operation

(71) Applicant: Worcester Polytechnic Institute, Worcester, MA (US)

(72) Inventors: Songbai JI, Westborough, MA (US); Shaoju WU, Quincy, MA (US)

(73) Assignee: Worcester Polytechnic Institute, Worcester, MA (US)

(21) Appl. No.: 18/598,664

(22) Filed: Mar. 7, 2024

Related U.S. Application Data
(60) Provisional application No. 63/450,842, filed on Mar. 8, 2023.

(51) Int. Cl. A61B 5/00; A61B 5/11

(52) U.S. Cl. CPC A61B 5/7275; A61B 5/11; A61B 5/682; A61B 5/7264; A61B 5/7425; A61B 2560/045; A61B 2560/0462; A61B 2562/0219

(57) ABSTRACT

A concussion detection system comprises a kinematic detection device configured to be carried by a head of a user and a concussion detection device disposed in electrical communication with the kinematic detection device. The concussion detection device comprises a controller configured to: receive kinematic data from the kinematic detection device, the kinematic data associated with a head impact of the user, apply the kinematic data to a strain prediction engine to generate a strain identifier associated with the head impact and a concussion risk assessment associated with the strain identifier, and output the concussion risk assessment based upon the strain identifier and configured to identify a concussion risk associated with the head impact. The concussion detection device can assess concussion risk in the user for an individual impact, or based on a history of multiple head impacts, and can further take into account head or brain size differences.

### RELATED APPLICATIONS

This patent application claims the benefit of U.S. Provisional Application No. 63/450,842, filed on Mar. 8, 2023, entitled “Deep-learning Model-based Concussion Detection System,” the contents and teachings of which are hereby incorporated by reference in their entirety.

### STATEMENT OF FEDERALLY SPONSORED RESEARCH

This invention was made with government support under Award Number 2114697 awarded by the National Science Foundation. The government has certain rights in the invention.

### BACKGROUND

Traumatic brain injury (TBI) is a leading, worldwide contributor to mortality and morbidity. TBI is typically initiated through an external head impact having relatively large and/or rapidly applied force that is sufficient to cause tissue damage. As such, impact forces or accelerations are considered to be the cause of brain injury, but not the best descriptor of tissue damage. Instead, there is general consensus that impact-induced brain strain, or the relatively rapid stretching of brain tissue, is the primary cause of TBI. However, given that real time and direct measurement of human brain deformation during a head impact is impractical, brain biomechanical models have been developed to estimate tissue deformation and, as a result, the occurrence of concussion and, if present, concussion severity.

Concussion detection models have been developed to produce detailed brain strains or strains in specific anatomical locations. For example, conventional neural networks can be trained using historical head impact data. The resulting concussion detection model is configured to predict brain strains associated with head impacts based upon head impact data provided to the model. The resulting brain strain can then be utilized to determine possible injury to the user, such as a concussion.

### SUMMARY

Conventional concussion detection models suffer from a variety of deficiencies. For example, head impact data can be applied to conventional concussion detection models to produce detailed brain strains or strains in specific anatomical locations. However, one challenge in applying the head impact data to the model relates to the computational efficiency in calculating strain. With conventional concussion detection models, it can take between about one hour to one day to calculate brain strain from head impact data. As such, the use of conventional concussion detection models in a real-world or real-time setting is not practical.

Further, while conventional concussion detection models provide information regarding brain tissue strain, these models fail to provide any information about strain changes in the brain over time. This can be impractical when analyzing head impact data for certain sports players. For example, football player can sustain dozens of head impacts over the course of a season. As such, an analysis of a head impact at the twelfth game of the season can be influenced by a head impact that occurred on the first game of the season.

By contrast to conventional concussion detection models, embodiments of the present innovation relate to a concussion detection system and method of operation. In one arrangement, a concussion detection system includes a concussion detection device, such as a computerized device having a controller (e.g., a memory and processor), disposed in electrical communication with a kinematic detection device. In one arrangement, the kinematic detection device can include impact sensors instrumented to provide kinematic data to the concussion detection device during operation. The concussion detection device is configured to apply kinematic data received from the kinematic detection device, such as received during an impact, to a strain prediction engine. As a result of such application, the concussion detection device is configured to generate strain identifiers, such as brain strain and strain rate, over the duration of the impact. Based upon the strain identifiers, the concussion detection device can generate an image representation of brain strain over time, as well as a concussion risk assessment, which can be utilized to facilitate concussion detection.

The concussion detection system can provide relatively rapid concussion detection following impact using both strain and strain rate, either of the whole brain or in specific brain regions and can be utilized in a variety of real-time scenarios. For example, the concussion detection system can be utilized by contact sports players, both professional and amateur, in schools, and for military personnel. Further, because of the configuration of the strain prediction engine, the concussion detection device, such as a laptop, tablet device, or mobile telecommunications device, can execute the model in a time efficient manner and does not require specialized personnel for operation.

A concussion detection system comprises a kinematic detection device configured to be carried by a head of a user and a concussion detection device disposed in electrical communication with the kinematic detection device. The concussion detection device comprises a controller having a memory and a processor, the controller configured to: receive kinematic data from the kinematic detection device, the kinematic data associated with a head impact of the user, apply the kinematic data to a strain prediction engine to generate a strain identifier associated with the head impact and a concussion risk assessment associated with the strain identifier, and output the concussion risk assessment based upon the strain identifier, the concussion risk assessment configured to identify a concussion risk associated with the head impact. The concussion detection device can assess concussion risk in the user for an individual impact, or based on a history of multiple head impacts, and can further take into account head or brain size differences.

A method for identifying concussion risk, comprises: receiving, by a concussion detection device, kinematic data from a kinematic detection device carried by a head of a user, the kinematic data associated with a head impact of a user; applying, by the concussion detection device, the kinematic data to a strain prediction engine to generate a strain identifier associated with the head impact and a concussion risk assessment associated with the strain identifier; and outputting, by the concussion detection device, the concussion risk assessment based upon the strain identifier, the concussion risk assessment configured to identify a concussion risk associated with the head impact.

A concussion detection device comprises a controller having a memory and a processor, the controller configured to: receive kinematic data from a kinematic detection device, the kinematic data associated with a head impact of the user; apply the kinematic data to a strain prediction engine to generate a strain identifier associated with the head impact and a concussion risk assessment associated with the strain identifier; and output the concussion risk assessment based upon the strain identifier, the concussion risk assessment configured to identify a concussion risk associated with the head impact.

### BRIEF DESCRIPTION OF THE DRAWINGS

The foregoing and other objects, features and advantages will be apparent from the following description of particular embodiments of the innovation, as illustrated in the accompanying drawings in which like reference characters refer to the same parts throughout the different views. The drawings are not necessarily to scale, emphasis instead being placed upon illustrating the principles of various embodiments of the innovation.

FIG. 1 illustrates a schematic representation of a concussion detection system, according to one arrangement.

FIG. 2 illustrates a schematic representation of a kinematic detection device carried by a user's head, according to one arrangement.

FIG. 3 illustrates a flowchart of a process performed by the concussion detection device of FIG. 1 when identifying concussion risk, according to one arrangement.

FIG. 4 illustrates a display of a voxelized brain image by the concussion detection device of FIG. 1, according to one arrangement

FIG. 5 illustrates a set of voxelized brain images over a duration of a user's head impact, according to one arrangement.

FIG. 6 illustrates a flowchart of a process performed by the concussion detection device of FIG. 1 when generating a concussion risk assessment, according to one arrangement.

FIG. 7 illustrates a schematic representation of a concussion detection system utilizing cranial dimension data, according to one arrangement.

FIG. 8A illustrates a schematic representation of cranial dimension data taken from a head of a user, according to one arrangement.

FIG. 8B illustrates a schematic representation of cranial dimension data taken from a brain of a user, according to one arrangement.

### DETAILED DESCRIPTION

Embodiments of the present innovation relate to a concussion detection system and method of operation. In one arrangement, a concussion detection system includes a concussion detection device, such as a computerized device having a controller (e.g., a memory and processor), disposed in electrical communication with a kinematic detection device. In one arrangement, the kinematic detection device can include impact sensors instrumented to provide kinematic data to the concussion detection device during operation. The concussion detection device is configured to apply kinematic data received from the kinematic detection device, such as received during an impact, to a strain prediction engine. As a result of such application, the concussion detection device is configured to generate strain identifiers, such as brain strain and strain rate, over the duration of the impact. Based upon the strain identifiers, the concussion detection device can generate an image representation of brain strain over time, as well as a concussion risk assessment, which can be utilized to facilitate concussion detection.

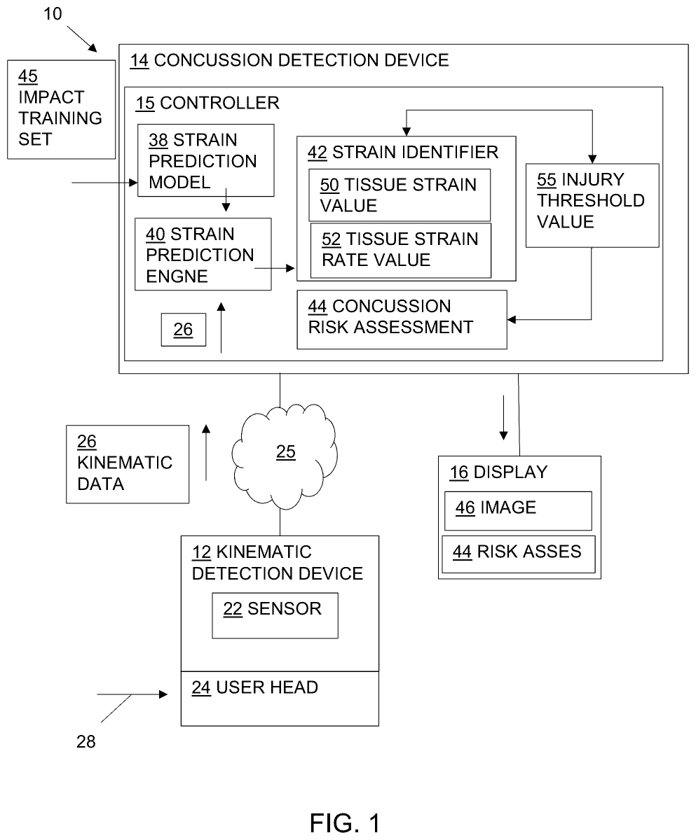

FIG. 1 illustrates a schematic representation of a concussion detection system 10, according to one arrangement. As illustrated, the concussion detection system 10 includes a kinematic detection device 12 disposed in electrical communication with a concussion detection device 14. In one arrangement, each of the kinematic detection device 12 and the concussion detection device 14 are configured as standalone devices disposed in electrical communication with each other. For example, the kinematic detection device 12 and the concussion detection device 14 can exchange electrical communications via a network 25 such as a local area network (LAN) or a wide area network (WAN). In one arrangement, the concussion detection system 10 includes both the kinematic detection device 12 and the concussion detection device 14 as part of a single device.

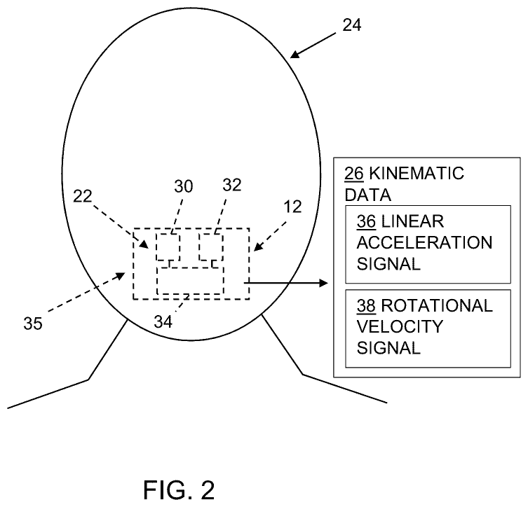

The kinematic detection device 12 is configured to be carried by a head 24 of a user and to generate signals associated with motion of a user's head 24, such as generated during an impact. The kinematic detection device 12 can be physically configured to be disposed on the user's head 24 in a variety of ways. For example, the kinematic detection device 12 can be configured as a helmet to be worn on the user's head 24, as an in-car device, or as a behind-the-car device. In one example, with additional reference to FIG. 2, the kinematic detection device 12 can be configured as a mouthguard 35. When placed within a user's mouth, the mouthguard 35 forms a relatively rigidly coupling with the user's upper denture. With such coupling, the mouthguard 35 allows associated sensors 22 to accurately generate kinematic data 26 based upon the motion of the user's head 24, such as following impact.

In one arrangement, the kinematic detection device 12 can be instrumented with a variety of sensors 22 configured to generate and provide kinematic data 26 to the concussion detection device 14 for further processing. For example, with reference to FIG. 2, the kinematic detection device 12 can include at least one linear accelerometer 30 configured to generate a linear acceleration signal 36 corresponding to the liner acceleration of the user's head 24 relative to the accelerometer 30, as resulting from an impact. The kinematic detection device can also include at least one gyroscope 32 configured generate a rotational velocity signal 38 corresponding to the rotational velocity of the user's head 24 relative to the gyroscope 32, as resulting from an impact.

Each sensor 22 is disposed in electrical communication with a transceiver 34 configured to transmit the linear acceleration signal 36 and the rotational velocity signal 38 to the concussion detection device 14 as kinematic data 26. The transceiver 34 can, in turn, be disposed in electrical communication with the concussion detection device 14 in a variety of ways. For example, the transceiver 34 can be configured to communicate with the concussion detection device 14 over the network 25 via a wireless communication protocol, such as the Bluetooth communication protocol.

Returning to FIG. 1, the concussion detection device 14 is configured as a computerized device, such as a personal computer, laptop, or tablet, and can include a controller 15, such as a processor and a memory. During operation, the concussion detection device 14 is configured to receive the kinematic data 26 from the kinematic detection device 12 in response to a user experiencing an impact load or force 28 and to generate and output a concussion risk assessment 44 based upon the kinematic data 26. For example, the concussion detection device 14 is configured to apply the received kinematic data 26 to a strain prediction engine 40, such as a deep-learning model, to generate strain identifiers 42 over the duration of the impact. Based upon the strain identifiers 42, the concussion detection device 14 can generate an image 46 representation of brain strain of the user over time for provision to a display 16, as well as the concussion risk assessment 44 which can be utilized to facilitate concussion detection.

In one arrangement, the concussion detection device 14 is configured to generate the strain prediction engine 40. As indicated in FIG. 1, the concussion detection device 14 includes a strain prediction model 38, such as one or more neural networks. The concussion detection device 14 can train the strain prediction model 38 using impact training data 45, such as simulated using the Worcester Head Injury Model, which is a physics-based finite element model. For example, based upon the application of impact loading data, the Worcester Head Injury Model simulation is configured to generate node-wise displacement from which element-wise strain can be derived.

In one arrangement, to increase the size of the training data set, the impact training data 45 can include augmented impact data that has been interpolated from the existing impact data. For example, during an augmentation process, a device operator can select an impact data element from the impact training data 45 where the impact data element is associated with an impact at a first brain location, such as the frontal lobe. The device operator can then assign the strain and strain rate values associated with that impact data element, to a second brain location, such as the temporal lobe, hereby creating an augmented impact data element. This augmented impact data element is then included with the impact training data 45 for training of the strain prediction model 38.

During a training operation, the concussion detection device 14 can retrieve the impact training data 45 from a database and apply the impact training data 45 to the strain prediction model 38. For example, the strain prediction model 38 can include both a transformer neural network and a separate convolutional neural network. The concussion detection device 14 can sequentially train the transformer neural network and convolutional neural network using the impact training data 45 to estimate five-dimensional (5D) spatiotemporal brain-skull relative displacement (e.g., a 3D voxelized image volume with two additional dimensions for displacement components and time, respectively) resulting from an impact load 28. With training of the strain prediction model 38, the concussion detection device 14 develops the trained strain prediction engine 40 which, in turn, is configured to receive real-time kinematic or impact data 26 (e.g., velocity and acceleration) and provide strain identifiers (e.g., brain strain and brain strain rate) by region of the brain (e.g., front, back, middle). It is noted that the concussion detection device 14 can continuously train the strain prediction model 38 over time with updated impact training data 45, such as retrieved from a database, to refine and update the strain prediction engine 40.

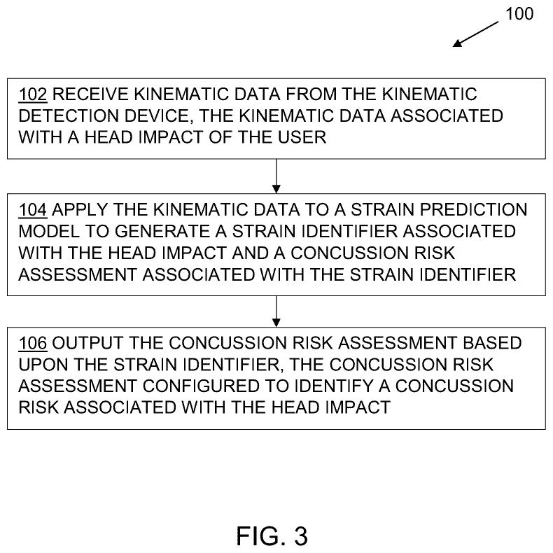

As provided above, during operation, the concussion detection device 14 is configured to predict a user's risk of concussion following an impact. FIG. 3 illustrates a flowchart 100 of a process performed by the concussion detection device 14 when identifying the concussion risk of a user, according to one arrangement.

In element 102, the concussion detection device 14 is configured to receive kinematic data 26 from a kinematic detection device 12, the kinematic data 26 associated with a head impact of a user. For example, as illustrated in FIG. 1, assume the case where the user is an athlete who participates in a contact sport, such as hockey or football. Prior to participating in the athletic event, the user would place the kinematic detection device 12 on the user's head 24. For example, in the case where the kinematic detection device 12 is configured as a mouthguard 35, the user would place the mouthguard 35 in his mouth.

As the user engages in the athletic event, if the user were to receive an impact load 28, such as an impact load 28 applied to the user's head 24, the impact load 28 can cause the sensors 22 of the kinematic detection device 12 to generate kinematic data 26 and provide the kinematic data 26 to the concussion detection device 14. For example, in response to motion of the user's head 24 resulting from the impact load 28, the linear accelerometer 30 can generate a linear acceleration signal 36 corresponding to the liner acceleration of the user's head 24 while the gyroscope 32 can generate a rotational velocity signal 38 corresponding to the rotational velocity of the user's head 24. A transceiver 34 of the kinematic detection device 12 can provide the linear acceleration signal 36 and the rotational velocity signal 38 to the concussion detection device 14 via network 25.

Returning to FIG. 3, in element 104, the concussion detection device 14 is configured to apply the kinematic data 26 to a strain prediction engine 40 to generate a strain identifier 42 associated with the head impact and a concussion risk assessment 44 associated with the strain identifier.

In one arrangement, with reference to FIG. 1, application of the kinematic data 26 to the strain prediction engine 40 can generate a strain identifier 42 by region of the user's brain (e.g., front, back, middle). While the strain identifier 42 can be configured in a variety of ways, in one arrangement the strain identifier 42 includes one or more tissue strain values 50 and one or more tissue strain rate value 52 for one or more brain regions of the user. For example, the tissue strain value 50 relates to the change in length or size of a portion of the user's brain relative to its initial length or size and provides information to global changes to the brain tissue of a user following an impact. The tissue strain rate value 52 relates to the change in strain of a portion of the user's brain over time also critical for microscale injury and provides information as to the deformation of individual axons of a user following impact.

The concussion detection device 14 can generate the concussion risk assessment 44 in a variety of ways. In one arrangement, the concussion detection device 14 is configured to compare the strain identifier 42 for various regions of the user's brain to an injury threshold value 55. In the case where the strain identifier 42 meets or exceeds the injury threshold value 55, the concussion detection device 14 is configured to generate the concussion risk assessment 44 which identifies the impact (e.g., the had impact) experienced by the user as being associated with a concussion. For example, assume that the injury threshold value 55 identifies a maximum tissue strain of 0.2 and a maximum tissue strain rate greater than 50 s⁻¹. Based on a comparison of the user's tissue strain value 50 and tissue strain rate value 52 with the injury threshold value 55, in the case where the concussion detection device 14 identifies the tissue strain value 50 being greater than or equal to 0.2 and/or the tissue strain rate value 52 as being greater than or equal to 50 s⁻¹, the concussion detection device 14 can generate a concussion risk assessment 44 that indicates the probable presence of a concussion in the user. Alternately, in the case where the concussion detection device 14 identifies the tissue strain value 50 as being less than 0.2 and/or the tissue strain rate value 52 as being less than 50 s⁻¹, the concussion detection device 14 can generate a concussion risk assessment 44 that indicates the probable absence of a concussion in the user.

Returning to FIG. 3, in element 104, the concussion detection device 14 is configured to output the concussion risk assessment 44 based upon the strain identifier 42, the concussion risk assessment 44 configured to identify a concussion risk associated with the head impact. In one arrangement, as illustrated in FIG. 1, the concussion detection device 14 can output the concussion risk assessment 44 to a display 16. By displaying the concussion risk assessment 44, a system operator can review the concussion risk assessment 44 of one or more users, such as an entire team of football or hockey players, and in the case where a concussion risk is identified, remove the user from further participation in the athletic activity. As such, the system operator can mitigate further injury to the user and can begin a concussion testing and/or treatment protocol for the user.

In traumatic brain injury, relatively high thresholds of either strain or strain rate can cause neurological damage. As such, the concussion detection system 10 is configured to provide relatively rapid concussion detection following impact using both strain 50 and strain rate 52, either of the whole brain or in specific brain regions, and can be utilized in a variety of real-time scenarios. For example, the concussion detection system can be utilized by contact sports players, both professional and amateur, in schools, and for military personnel. Further, because of the configuration of the strain prediction engine 40, the concussion detection device 14, such as a conventional laptop, tablet device, or mobile telecommunications device, can execute the model 40 in a time efficient manner and does not require specialized personnel for operation.

As provided above, the concussion detection device 14 is configured to output the concussion risk assessment 44 based upon predicted strain identifiers 42 associated with a user receiving an impact loading 28. In one arrangement, as shown in FIG. 1, the concussion detection device 14 can also output a brain image 46 identifying a predicted displacement field and the corresponding tissue strain value 50 and tissue strain rate value 52 associated with the brain region of the head. A system operator can use the brain image 46 to better understand the concussion risk assessment 44 provided by the concussion detection device 14 and to develop a concussion testing and/or treatment protocol for the user.

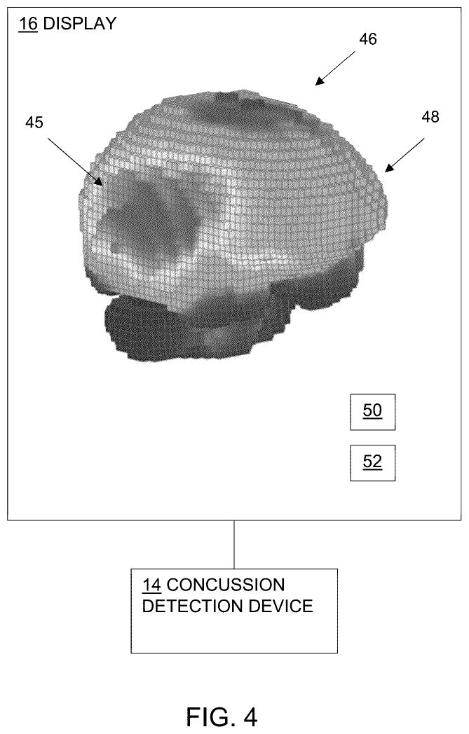

The brain image 46 can be configured in a variety of ways. For example, as illustrated in FIG. 4, the brain image 46 is configured as a voxelzed image 48 which can identify a predicted displacement field 45 and three-dimensional strains of a user's brain by region. For example, as illustrated, the voxelzed image 48 identifies a displacement field 45 in the frontal lobe of the user's brain, along with corresponding three-dimensional tissue strain values 50 and tissue strain rate values 52. By presenting the brain image 46 as a voxelzed image 48, the concussion detection device 14 reduces the amount of data used to generate the brain image 46, such as relative to finite element (FE) images. Because the voxelized image 48 utilizes a relatively minimal amount of data, the concussion detection device 14 can generate an output the voxelzed image 48 in a relatively fast manner, thereby allowing the system operator to receive a user's brain image 46 following an impact load 28 in substantially real time. Further, by generating the brain image 46 as a voxelzed image 48, the concussion detection device 14 can provide the brain image 46 in a standard image format which facilitates multimodal analysis as well as which allows rapid data sharing among networked concussion detection devices 14 and/or other system operators.

In one arrangement, the concussion detection device 14 can also output brain images 46 over an impact duration, such as the time interval when the impact load 28 is applied to the user. As such, the concussion detection device 14 can show the change in strain data of the user's brain over time (e.g., the temporal domain), such as from beginning of an impact until the end of the impact,

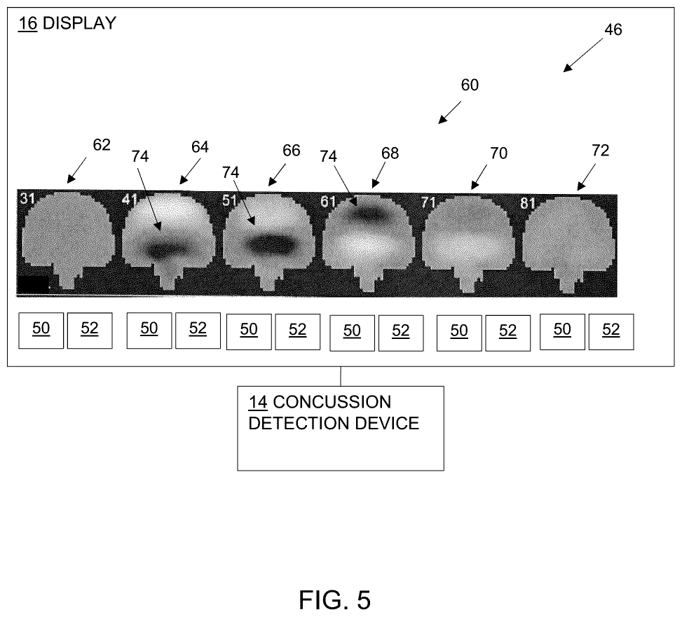

With reference to FIG. 5, when outputting the brain image 46, such as to the display 16, the concussion detection device 14 is configured to output a consecutive set of brain images 60, such as voxelized brain images, where each brain image 62 through 72 of the consecutive set of brain images 60 is associated with a corresponding time point of a set of time points during the head impact. For example, as shown, the concussion detection device 14 can generate and display a first image 62 at 31 milliseconds (ms) into the impact, a second image 64 at 41 ms into the impact, and onward until the last image is displayed 72 at the conclusion of the impact. It is noted that while the images 62 through 72 are presented collectively, the concussion detection device 14 can provide the images 62 through 72 to the display 16 in a consecutive manner, thereby animating the position of the predicted displacement field 74 within the user's brain during the impact duration. Additionally, the concussion detection device 14 can generate and display the corresponding tissue strain value 50 and tissue strain rate value 52 associated with the brain region of the head at each corresponding time point.

By displaying the consecutive set of brain images 60 identifying the predicted displacement field 74 and the corresponding tissue strain values 50 and tissue strain rate values 52, the concussion detection device 14 provides a dynamic representation of the user's brain responses during an impact. As such, the concussion detection device 14 allows the system operator to better understand the predicted brain reaction to an impact loading 28 over the duration of the impact, in order to select an optimal concussion testing and/or treatment protocol for the user.

As provided above, when generating a concussion risk assessment 44, the concussion detection device 14 is configured to compare the strain identifier 42 for various regions of the user's brain to an injury threshold value 55. In the case where the concussion detection device 14 identifies the tissue strain value 50 and/or the tissue strain rate value 52 as being less than the injury threshold value 55, the concussion detection device 14 can generate a concussion risk assessment 44 that indicates the probable absence of a concussion in the user. However, in certain cases, a user, such as a football player may experience a relatively large number of head impacts over his career where no one single impact produces a tissue strain value 50 or tissue strain rate value 52 strain exceeds the injury threshold 55. Additionally, the user may have experienced repeated head impacts that generate these sub-threshold tissue strain values 50 or tissue strain rate values 52 in a particular brain region. If a particular region has experienced a number of sub-threshold strains and or strain rates over time (e.g., over a season or a career), this may be indicative of brain injury. Accordingly, in one arrangement, the concussion detection device 14 can be configured to take into account the history of the user, such as a career athlete, when generating the concussion risk assessment 44.

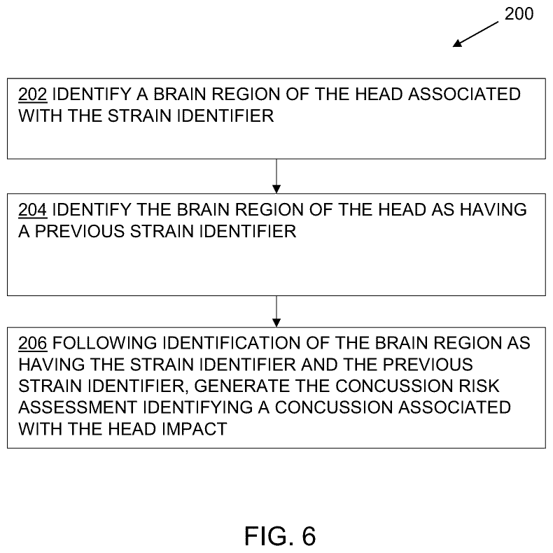

For example, FIG. 6 illustrates a flowchart 200 of a process performed by the concussion detection device 14 following the comparison of a strain identifier 42 to an injury threshold value 55 where the detection of the strain identifier 42 falls below the injury threshold value 55.

In element 202, the concussion detection device 14 is configured to identify a brain region of the head 24 associated with the strain identifier 42. For example, as provided above, in response to receiving an impact load 28, the kinematic detection device 12 generates and provides kinematic data 26 to the concussion detection device 14. The concussion detection device 14 applies the kinematic data 26 to the strain prediction engine 40 to generate predicted tissue strain values 50 and tissue strain rate values 52 for one or more regions of the user's brain. As such, each strain identifier 42 can include one or more associated brain region identifiers for a given user.

In element 204, the concussion detection device 14 is configured to identify the brain region of the head 24 as having a previous strain identifier. For example, the concussion detection device 14 can identify the user who has generated the kinematic data 26, such as via a user identifier associated with the kinematic data 26. The concussion detection device 14 is then configured to access historical strain identifier information, such as stored in a historical database, and to identify every instance of a user record within the database based upon the user identifier. In one arrangement, the historical database can store previously generated strain identifiers and associated brain region information which has been accumulated over some time duration (e.g., game, series of gams, user's career, etc.). For each user record associated with the user, the concussion detection device 14 is configured to identify the historic brain region data associated with each historical strain identifier stored for the user and to compare the brain region identifiers of the current (e.g., recently generated) strain identifier 42 with the identified historic brain region data.

In element 206, following identification of the brain region as having the strain identifier 42 and the previous strain identifier, the concussion detection device 14 is configured to generate the concussion risk assessment 44 identifying a concussion associated with the head impact 28. For example, assume the case where the historical strain identifier information identifies the user as having several head impacts which generated sub-threshold strains and strain rates in the frontal lobe of the user's brain. In the case where the concussion detection device 14 detects the strain identifier 42 as also being associated with the frontal lobe of the user's brain, the concussion detection device 14 can generate a concussion risk assessment 44 that indicates the probable presence of a concussion in the user and can provide a recommendation that the user refrain from further participation in the athletic event.

As indicated above, the concussion detection device 14 is configured to train a strain prediction model 38 to generate a strain prediction engine 40 and to utilize the strain prediction engine 40 to predict a user's risk of concussion following an impact. However, the size of each user's head can be different. For example, a male head can typically be larger than a female head. In another example, the head of a young adult can typically be larger than that of a pre-teenager. In one arrangement, in order to increase the accuracy of the predicted strain identifiers 42 and concussion risk assessments 44 for a user, the concussion detection device 14 can be configured to take into account the cranial sizes of users to make the predicted strain identifiers 42 and resulting concussion risk assessments 44 more personalized and accurate.

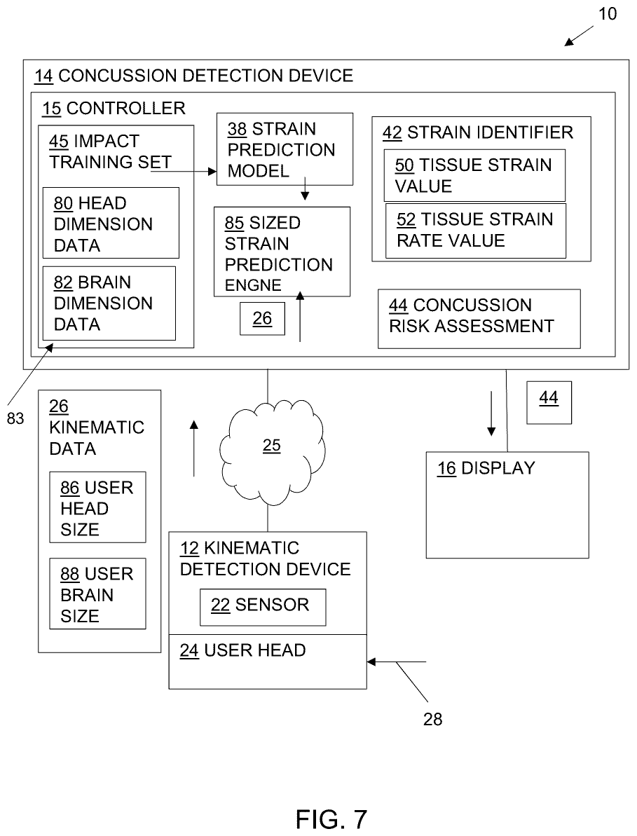

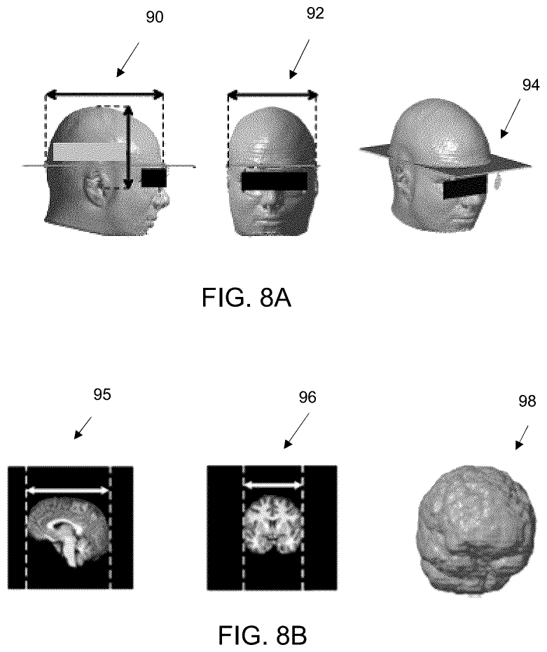

With reference to FIG. 7, when the concussion detection device 14 trains the strain prediction model 38, in order to account for variation in head size, the impact training set 45 can include cranial size data 83 for each simulated impact. For example, the cranial size data 83 can include head dimension data 80, such as measured from a user's head. While the head dimension data 80 can be configured in a variety of ways in one arrangement, with reference to FIG. 8A, the head dimension data 80 includes head length 90, head width 92, and head circumference 94 data measured from a user's head. In another example, the cranial size data 83 can include brain dimension data 82, such as measured from a user's brain using an imaging technique such as magnetic resonance imaging. While the brain dimension data 82 can be configured in a variety of ways in one arrangement, with reference to FIG. 8B, the brain dimension data 82 includes brain length 95, brain width 96, and brain volume 98 data measured from a user's brain. During the training process, the concussion detection device 14 can apply the cranial size data 83 to the x-y-z directions (e.g., either of the head dimension data 80 or the brain dimension data 82) of the strain prediction model 38 as a scaling factor to generate a sized strain prediction engine 85.

During operation, prior to use, a user can have either his head size measured (e.g., head length 90, head width 92, and head circumference 94) or his brain size measured (e.g., brain length 95, brain width 96, and brain volume 98) which can be included within the kinematic detection device 12, for example, as either user head size data 86 or user brain size data 86, respectively. In the event that the user receives an impact load 28, the kinematic detection device can transmit resulting kinematic data 26 generated by the kinematic detection device 12, along with either the user head size data 86 or user brain size data 86, to the concussion detection device 14. Upon receipt, the concussion detection device 14 can apply the kinematic data 26, along with either the user head size data 86 or user brain size data 86, to the sized strain prediction engine 85. Such application results in a strain identifier 42 having predicted tissue strain values 50 and tissue strain rate values 52 which are more tailored to user's individual head or brain size. As such, by taking a user's head or brain size into account, the concussion detection device 14 can generate a customized concussion risk assessment 44 for a particular user.

While various embodiments of the innovation have been particularly shown and described, it will be understood by those skilled in the art that various changes in form and details may be made therein without departing from the spirit and scope of the innovation as defined by the appended claims.

What is claimed is:

1. A concussion detection system, comprising:
a kinematic detection device configured to be carried by a head of a user; and
a concussion detection device disposed in electrical communication with the kinematic detection device, the concussion detection device comprising a controller having a memory and a processor, the controller configured to:
receive kinematic data from the kinematic detection device, the kinematic data associated with a head impact of the user,
apply the kinematic data to a strain prediction engine to generate a strain identifier associated with the head impact and a concussion risk assessment associated with the strain identifier, and
output the concussion risk assessment based upon the strain identifier, the concussion risk assessment configured to identify a concussion risk associated with the head impact.

2. The concussion detection system of claim 1, wherein the kinematic detection device comprises:
at least one accelerometer configured to generate a linear acceleration signal;
at least one gyroscope configured to generate a rotational velocity signal; and
a transceiver disposed in electrical communication with the at least one accelerometer and the at least one gyroscope, the transceiver configured to transmit the linear acceleration signal and the rotational velocity signal as kinematic data to the concussion detection device.

3. The concussion detection system of claim 2, wherein the kinematic detection device comprises a mouthguard.

4. The concussion detection system of claim 1, wherein when generating the strain identifier associated with the head impact, the controller is configured to generate a tissue strain value and a tissue strain rate value by a brain region of the user.

5. The concussion detection system of claim 4, wherein in response to generating the tissue strain value and the tissue strain rate value for brain region of the head, the controller is configured to output a brain image identifying a predicted displacement field and the corresponding tissue strain value and tissue strain rate value associated with the brain region of the head.

6. The concussion detection system of claim 5, wherein when outputting the brain image identifying the predicted displacement field and the corresponding tissue strain value and tissue strain rate value associated with the brain region of the head, the controller is configured to output a consecutive set of brain images;
each brain image of the consecutive set of brain images associated with a corresponding time point of a set of time points during the head impact; and
each brain image of the consecutive set of brain images identifying the predicted displacement field and the corresponding tissue strain value and tissue strain rate value associated with the brain region of the head at the corresponding time point.

7. The concussion detection system of claim 1, wherein, when generating the concussion risk assessment associated with the strain identifier, the controller is configured to:
compare the strain identifier to an injury threshold value; and
when the strain identifier meets the injury threshold value, generate the concussion risk assessment identifying a concussion associated with the head impact.

8. The concussion detection system of claim 1, wherein, when generating the concussion risk assessment associated with the strain identifier, the controller is configured to:
compare the strain identifier to an injury threshold value; and
when the strain identifier falls below the injury threshold value:
identify a brain region of the head associated with the strain identifier,
identify the brain region of the head as having a previous strain identifier, and
following identification of the brain region as having the strain identifier and the previous strain identifier, generate the concussion risk assessment identifying a concussion associated with the head impact.

9. The concussion detection system of claim 1, wherein:
when receiving kinematic data from the kinematic detection device, the controller is configured to further receive head size data associated with the head of the user; and
when applying the kinematic data to the strain prediction engine to generate the strain identifier associated with the head impact and the concussion risk assessment associated with the strain identifier, the controller is configured to apply the kinematic data and the head size data to the strain prediction engine to generate the strain identifier associated with the head impact and the concussion risk assessment associated with the strain identifier.

10. The concussion detection system of claim 1, wherein:
when receiving kinematic data from the kinematic detection device, the controller is configured to further receive brain size data associated with the head of the user; and
when applying the kinematic data to the strain prediction engine to generate the strain identifier associated with the head impact and the concussion risk assessment associated with the strain identifier, the controller is configured to apply the kinematic data and the brain size data to the strain prediction engine to generate the strain identifier associated with the head impact and the concussion risk assessment associated with the strain identifier.

11. A method for identifying concussion risk, comprising:
receiving, by a concussion detection device, kinematic data from a kinematic detection device carried by a head of a user, the kinematic data associated with a head impact of a user;
applying, by the concussion detection device, the kinematic data to a strain prediction engine to generate a strain identifier associated with the head impact and a concussion risk assessment associated with the strain identifier; and
outputting, by the concussion detection device, the concussion risk assessment based upon the strain identifier, the concussion risk assessment configured to identify a concussion risk associated with the head impact.

12. The method of claim 11, wherein receiving kinematic data from a kinematic detection device comprises:
receiving, by the concussion detection device, a linear acceleration signal generated by at least one accelerometer carried by the kinematic detection device; and
receiving, by the concussion detection device, a rotational velocity signal generated by at least one gyroscope carried by the kinematic detection device.

13. The method of claim 12, wherein the kinematic detection device comprises a mouthguard.

14. The method of claim 11, wherein generating the strain identifier associated with the head impact comprises generating, by the concussion detection device, a tissue strain value and a tissue strain rate value by brain region of the user.

15. The method of claim 14, wherein generating the tissue strain value and the tissue strain rate value for brain region of the head further comprises outputting, by the concussion detection device, a brain image identifying a predicted displacement field and the corresponding tissue strain value and tissue strain rate value associated with the brain region of the head.

16. The method of claim 15, wherein outputting the brain image identifying the predicted displacement field and the corresponding tissue strain value and tissue strain rate value associated with the brain region of the head comprises outputting, by the concussion detection device, a consecutive set of brain images;
each brain image of the consecutive set of brain images associated with a corresponding time point of a set of time points during the head impact; and
each brain image of the consecutive set of brain images identifying the predicted displacement field and the corresponding tissue strain value and tissue strain rate value associated with the brain region of the head at the corresponding time point.

17. The method of claim 11, wherein generating the concussion risk assessment associated with the strain identifier comprises:
comparing, by the concussion detection device, the strain identifier to an injury threshold value; and
when the strain identifier meets the injury threshold value, generating, by the concussion detection device, the concussion risk assessment identifying a concussion associated with the head impact.

18. The method of claim 11, wherein generating the concussion risk assessment associated with the strain identifier comprises:
comparing, by the concussion detection device, the strain identifier to an injury threshold value; and
when the strain identifier falls below the injury threshold value:
identifying, by the concussion detection device, a brain region of the head associated with the strain identifier,
identifying, by the concussion detection device, the brain region of the head as having a previous strain identifier, and
following identification of the brain region as having the strain identifier and the previous strain identifier, generating, by the concussion detection device, the concussion risk assessment identifying a concussion associated with the head impact.

19. The method of claim 11, wherein:
receiving kinematic data from the kinematic detection device comprises further receiving, by the concussion detection device, head size data associated with the head of the user; and
applying the kinematic data to the strain prediction engine to generate the strain identifier associated with the head impact and the concussion risk assessment associated with the strain identifier comprises applying, by the concussion detection device, the kinematic data and the head size data to the strain prediction engine to generate the strain identifier associated with the head impact and the concussion risk assessment associated with the strain identifier.

20. The method of claim 11, wherein:
receiving kinematic data from the kinematic detection device comprises further receiving, by the concussion detection device, brain size data associated with the head of the user; and
applying the kinematic data to the strain prediction engine to generate the strain identifier associated with the head impact and the concussion risk assessment associated with the strain identifier comprises applying, by the concussion detection device, the kinematic data and the brain size data to the strain prediction engine to generate the strain identifier associated with the head impact and the concussion risk assessment associated with the strain identifier.

21. A concussion detection device comprises a controller having a memory and a processor, the controller configured to:
receive kinematic data from a kinematic detection device, the kinematic data associated with a head impact of the user;
apply the kinematic data to a strain prediction engine to generate a strain identifier associated with the head impact and a concussion risk assessment associated with the strain identifier; and
output the concussion risk assessment based upon the strain identifier, the concussion risk assessment configured to identify a concussion risk associated with the head impact.
